(19) United States
(12) Patent Application Publication
(10) Pub. No.: US 2021/0265064 A1
(43) Pub. Date: Aug. 26, 2021

(54) SYSTEM AND METHOD FOR PROVIDING MODEL-BASED PREDICTIONS OF QUALITY OF LIFE IMPLICATIONS OF A TREATMENT VIA INDIVIDUAL-SPECIFIC MACHINE LEARNING MODELS

(71) Applicant: KONINKLIJKE PHILIPS N.V., Eindhoven (NL)

(72) Inventors: Monique HENDRIKS, Eindhoven (NL); Jacek Lukasz KUSTRA, Eindhoven (NL); Sandra VOSBERGEN, Liempde (NL)

(73) Assignee: Koninklijke Philips N.V., Eindhoven (NL)

(21) Appl. No.: 17/053,954

(22) PCT Filed: May 8, 2019
(86) PCT No.: PCT/EP2019/061876

§ 371 Date: Nov. 9, 2020

Related U.S. Application Data
(60) Provisional application No. 62/668,348, filed on May 8, 2018.

(51) Int. Cl. G16H 50/70; G06N 20/00; G16H 50/20; G06Q 50/00; G16H 10/60; G16H 50/30; G06Q 30/02; H04L 29/08

(52) U.S. Cl. CPC G16H 50/70; G06N 20/00; G16H 50/20; G06Q 50/01; G16H 40/20; G16H 50/30; G06Q 30/0201; H04L 67/22; G16H 10/60

(57) ABSTRACT

The present disclosure pertains to a system for providing model-based predictions of quality of life implications of a treatment via individual-specific machine learning models. In some embodiments, the system (i) obtains social media information related to an individuals social media activities, the individual awaiting prescription of a predetermined treatment; (ii) performs one or more queries to obtain health and social media information associated with similar individuals having (a) similar social media activities and (b) a record of receiving the predetermined treatment; (iii) provides the health and social media information associated with the similar individuals to a machine learning model to train the machine learning model; and (iv) provides the social media information associated with the individual to the machine learning model to predict (a) a likelihood of the one or more health states and (b) an estimated importance of each of the one or more health states for the individual.

### BACKGROUND

### 1. Field

The present disclosure pertains to a system and method for providing model-based predictions of quality of life implications of a treatment via individual-specific machine learning models.

### 2. Description of the Related Art

Decision support models in health care may be used for determining recommended treatments for a patient. The treatment suggested by a decision support model may optimize survival, quality of life, cost-effectiveness, or a combination thereof. Although automated and other computer-assisted treatment recommendation systems exist, such systems may often disregard treatment consequences extending beyond physical health states and including mental, emotional, or social functioning. For example, while current treatment recommendation systems may define the concept of quality of life (e.g., via questionnaires), the use of this concept in practice may be limited to population measures. On the individual level, these concepts may not be concrete and detailed enough to be used to determine the best care path for the patient that incorporates the affective impact of a treatment on the patient's life. These and other drawbacks exist.

### SUMMARY

Accordingly, one or more aspects of the present disclosure relate to a system for providing model-based predictions of quality of life implications of a treatment via individual-specific machine learning models. The system comprises one or more processors configured by machine readable instructions and/or other components. The one or more processors are configured to: obtain, from one or more databases, social media information related to an individual's social media activities, the individual awaiting prescription of a predetermined treatment; perform one or more queries based on the predetermined treatment and the social media information associated with the individual to obtain health and social media information associated with similar individuals having (i) similar social media activities and (ii) a record of receiving the predetermined treatment, the health and social media information indicating one or more health states experienced by the similar individuals respectively subsequent to the similar individuals receiving the predetermined treatment; provide the health and social media information associated with the similar individuals to a machine learning model to train the machine learning model; and provide, subsequent to the training of the machine learning model, the social media information associated with the individual to the machine learning model to predict (a) a likelihood of the one or more health states and (b) an estimated importance of each of the one or more health states for the individual.

Another aspect of the present disclosure relates to a method for providing model-based predictions of quality of life implications of a treatment via individual-specific machine learning models with a system. The system comprises one or more processors configured by machine readable instructions and/or other components. The method comprises: obtaining, with one or more processors, social media information related to an individual's social media activities from one or more databases, the individual awaiting prescription of a predetermined treatment; performing, with the one or more processors, one or more queries based on the predetermined treatment and the social media information associated with the individual to obtain health and social media information associated with similar individuals having (i) similar social media activities and (ii) a record of receiving the predetermined treatment, the health and social media information indicating one or more health states experienced by the similar individuals respectively subsequent to the similar individuals receiving the predetermined treatment; providing, with the one or more processors, the health and social media information associated with the similar individuals to a machine learning model to train the machine learning model; and providing, with the one or more processors, the social media information associated with the individual to the machine learning model subsequent to the training of the machine learning model to predict (a) a likelihood of the one or more health states and (b) an estimated importance of each of the one or more health states for the individual.

Still another aspect of present disclosure relates to a system for providing model-based predictions of quality of life implications of a treatment via individual-specific machine learning models. The system comprises: means for obtaining social media information related to an individual's social media activities from one or more databases, the individual awaiting prescription of a predetermined treatment; means for performing one or more queries based on the predetermined treatment and the social media information associated with the individual to obtain health and social media information associated with similar individuals having (i) similar social media activities and (ii) a record of receiving the predetermined treatment, the health and social media information indicating one or more health states experienced by the similar individuals respectively subsequent to the similar individuals receiving the predetermined treatment; means for providing the health and social media information associated with the similar individuals to a machine learning model to train the machine learning model; and means for providing the social media information associated with the individual to the machine learning model subsequent to the training of the machine learning model to predict (a) a likelihood of the one or more health states and (b) an estimated importance of each of the one or more health states for the individual.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system configured for providing model-based predictions of quality of life implications of a treatment via individual-specific machine learning models.

FIG. 2 illustrates processing the digital footprint of similar individuals, in accordance with one or more embodiments.

FIG. 3 illustrates analysis of post-treatment affect and health outcomes of similar individuals, in accordance with one or more embodiments.

FIG. 4 illustrates predicting treatment impacts via a trained machine learning model, in accordance with one or more embodiments.

FIG. 5 illustrates estimating the importance of each impact via a trained machine learning model, in accordance with one or more embodiments.

FIG. 6 illustrates ranking health states based on a combination of likelihood of occurrence and estimated importance, in accordance with one or more embodiments.

FIG. 7 illustrates a method for providing model-based predictions of quality of life implications of a treatment via individual-specific machine learning models, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

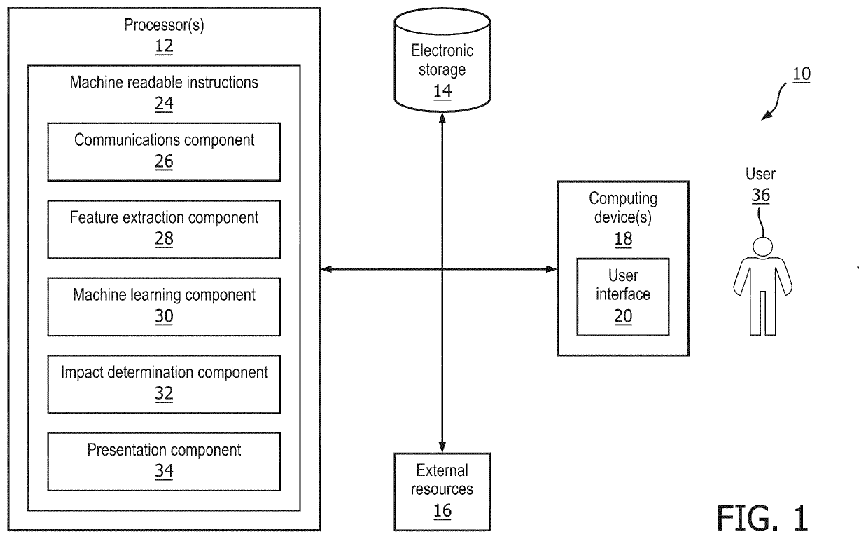

FIG. 1 is a schematic illustration of a system 10 configured for providing model-based predictions of quality of life implications of a treatment via individual-specific machine learning models. In some embodiments, system 10 is configured to obtain pre-treatment data related to a patient's health records, digital footprint (e.g., available data on the patient such as google-hits, posts on social media and forums, etc.), or other information. In some embodiments, system 10 is configured to determine (e.g., via machine learning models, natural language processing, etc.), from the digital footprint, one or more personality characteristics, personal goals and values, or other attributes of the patient. In some embodiments, system 10 is configured to obtain health and social media information associated with similar individuals having (i) similar digital footprint and (ii) a record of receiving the treatment. In some embodiments, the health information associated with the similar individuals indicates health states, such as survival, side effects, patient reported outcomes, emotional stability (or other mental effects), or other information. In some embodiments, the social media information associated with the similar individuals indicates one or more personality characteristics, personal goals and values, or other attributes associated with the similar individuals. In some embodiments, system 10 determines, from the similar individuals' digital footprint, post-treatment affective information. In some embodiments, post-treatment affective information includes post-treatment anxiety, ability to execute certain activities, ease with which the inability to execute certain activities was accepted, reorientation of life goals where necessary, or other information. In some embodiments, system 10 is configured to provide the health and social media information associated with similar individuals as input to a machine learning model to train the machine learning model. In some embodiments, system 10 is configured to provide, subsequent to the training of the machine learning model, the digital footprint associated with the patient to the machine learning model to generate one or more predictions related to a likelihood of the health states, predictions related to an estimated importance of each of the health states for the patient, or other predictions. In some embodiments, system 10 is configured such that derived features of past patients are used to construct a list of all possible future health states after each treatment option (e.g., health states related physical, emotional, or social well-being after each treatment option). In some embodiments, system 10 is configured to provide, through comparison of the patient's digital footprint with digital footprint of already treated individuals, an estimation related to a level of impact of each possible future health state on the patient. In some embodiments, system 10 is configured to determine, based on a predicted probability of each future health state's occurrence for the patient, a ranking of one or more future health states with respect to the treatment. In some embodiments, system 10 may determine that additional information is needed to increase the accuracy of such prediction or ranking. As an example, based on the comparison of the patient's digital footprint with digital footprint of already treated individuals, system 10 may determine one or more features missing from the patient's digital footprint (e.g., that are indicated in the digital footprint of already treated individuals), and provide an indication of such missing features for presentation to a user (e.g., the patient, a caregiver, an administrator, or other user so that the missing features can be obtained from the patient or other sources).

In some embodiments, system 10 is configured to generate one or more predictions related to the likelihood of the health states (e.g., health states related physical, emotional, or social well-being, predictions related to the estimated importance of each of the health states for the patient, etc., or perform other operations described herein via one or more prediction models. Such prediction models may include neural networks, other machine learning models, or other prediction models. As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

In some embodiments, system 10 comprises processors 12, electronic storage 14, external resources 16, computing device 18 (e.g., associated with user 36), or other components.

Electronic storage 14 comprises electronic storage media that electronically stores information (e.g., social media information associated with the individual or the similar individuals, health information associated with the similar individuals). The electronic storage media of electronic storage 14 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 14 may be (in whole or in part) a separate component within system 10, or electronic storage 14 may be provided (in whole or in part) integrally with one or more other components of system 10 (e.g., computing device 18, etc.). In some embodiments, electronic storage 14 may be located in a server together with processors 12, in a server that is part of external resources 16, and/or in other locations. Electronic storage 14 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 14 may store software algorithms, information determined by processors 12, information received via processors 12 and/or graphical user interface 20 and/or other external computing systems, information received from external resources 16, and/or other information that enables system 10 to function as described herein.

External resources 16 include sources of information and/or other resources. For example, external resources 16 may include a population's electronic medical record (EMR), the population's electronic health record (EHR), or other information. In some embodiments, external resources 16 include health information related to the population. In some embodiments, the health information comprises demographic information, vital signs information, medical condition information indicating medical conditions experienced by individuals in the population, treatment information indicating treatments received by the individuals, care management information, and/or other health information. In some embodiments, external resources 16 include sources of information such as databases, websites, etc., external entities participating with system 10 (e.g., a medical records system of a health care provider that stores medical history information of patients, publicly and privately accessible social media websites), one or more servers outside of system 10, and/or other sources of information. In some embodiments, external resources 16 include components that facilitate communication of information such as a network (e.g., the internet), electronic storage, equipment related to Wi-Fi technology, equipment related to Bluetooth® technology, data entry devices, sensors, scanners, and/or other resources. In some embodiments, some or all of the functionality attributed herein to external resources 16 may be provided by resources included in system 10.

Processors 12, electronic storage 14, external resources 16, computing device 18, and/or other components of system 10 may be configured to communicate with one another, via wired and/or wireless connections, via a network (e.g., a local area network and/or the internet), via cellular technology, via Wi-Fi technology, and/or via other resources. It will be appreciated that this is not intended to be limiting, and that the scope of this disclosure includes embodiments in which these components may be operatively linked via some other communication media. In some embodiments, processors 12, electronic storage 14, external resources 16, computing device 18, and/or other components of system 10 may be configured to communicate with one another according to a client/server architecture, a peer-to-peer architecture, and/or other architectures.

Computing device 18 may be configured to provide an interface between user 36 and/or other users, and system 10. In some embodiments, computing device 18 is and/or is included in desktop computers, laptop computers, tablet computers, smartphones, smart wearable devices including augmented reality devices (e.g., Google Glass), wrist-worn devices (e.g., Apple Watch), and/or other computing devices associated with user 36, and/or other users. In some embodiments, computing device 18 facilitates presentation of a list of individuals assigned to a care manager, or other information. Accordingly, computing device 18 comprises a user interface 20. Examples of interface devices suitable for inclusion in user interface 20 include a touch screen, a keypad, touch sensitive or physical buttons, switches, a keyboard, knobs, levers, a camera, a display, speakers, a microphone, an indicator light, an audible alarm, a printer, tactile haptic feedback device, or other interface devices. The present disclosure also contemplates that computing device 18 includes a removable storage interface. In this example, information may be loaded into computing device 18 from removable storage (e.g., a smart card, a flash drive, a removable disk, etc.) that enables caregivers or other users to customize the implementation of computing device 18. Other exemplary input devices and techniques adapted for use with computing device 18 or the user interface include an RS-232 port, RF link, an IR link, a modem (telephone, cable, etc.), or other devices or techniques.

Processor 12 is configured to provide information processing capabilities in system 10. As such, processor 12 may comprise one or more of a digital processor, an analog processor, a digital circuit designed to process information, an analog circuit designed to process information, a state machine, or other mechanisms for electronically processing information. Although processor 12 is shown in FIG. 1 as a single entity, this is for illustrative purposes only. In some embodiments, processor 12 may comprise a plurality of processing units. These processing units may be physically located within the same device (e.g., a server), or processor 12 may represent processing functionality of a plurality of devices operating in coordination (e.g., one or more servers, computing device, devices that are part of external resources 16, electronic storage 14, or other devices.)

As shown in FIG. 1, processor 12 is configured via machine-readable instructions 24 to execute one or more computer program components. The computer program components may comprise one or more of a communications component 26, a feature extraction component 28, a machine learning component 30, an impact determination component 32, a presentation component 34, or other components. Processor 12 may be configured to execute components 26, 28, 30, 32, or 34 by software; hardware; firmware; some combination of software, hardware, or firmware; or other mechanisms for configuring processing capabilities on processor 12.

It should be appreciated that although components 26, 28, 30, 32, and 34 are illustrated in FIG. 1 as being co-located within a single processing unit, in embodiments in which processor 12 comprises multiple processing units, one or more of components 26, 28, 30, 32, or 34 may be located remotely from the other components. The description of the functionality provided by the different components 26, 28, 30, 32, or 34 described below is for illustrative purposes, and is not intended to be limiting, as any of components 26, 28, 30, 32, or 34 may provide more or less functionality than is described. For example, one or more of components 26, 28, 30, 32, or 34 may be eliminated, and some or all of its functionality may be provided by other components 26, 28, 30, 32, or 34. As another example, processor 12 may be configured to execute one or more additional components that may perform some or all of the functionality attributed below to one of components 26, 28, 30, 32, or 34.

In some embodiment, the present disclosure comprises means for obtaining, from one or more databases (e.g., electronic storage 14, external resources 16, etc.), social media information related to an individual's social media activities. In some embodiments, such means for obtaining takes the form of communications component 26. In some embodiments, the individual may be awaiting prescription of a predetermined treatment (e.g., for a particular diagnosis). In some embodiments, the predetermined treatment is selected from one or more available and/or recommended treatments for the particular diagnosis. In some embodiments, the individual's social media activities include one or more of Facebook likes, Facebook posts, Tweets, Retweets, accounts followed on Twitter, people followed on LinkedIn, browsing data, forum posts, or other social media activities.

In some embodiments, communications component 26 is configured to obtain a data collection representative of a population of individuals having a record of receiving the predetermined treatment. In some embodiments, the data collection may be representative of 100 or more individuals, 1,000 or more individuals, 10,000 or more individuals, 100,000 or more individuals, 1,000,000 or more individuals, 100,000,000 or more individuals, or other number of individuals. In some embodiments, the data collection may include health and social media information corresponding to the individuals. In some embodiments, social media information associated with the population of individuals includes the individuals' social media activities (e.g., Facebook likes, Facebook posts, Tweets, Retweets, accounts followed on Twitter, people followed on LinkedIn, browsing data, public forum posts, private e-mails, chat messages, activities on closed forums, or other social media activities). In some embodiments, the health information corresponding to the individuals includes one or more health states experienced by the similar individuals respectively subsequent to the similar individuals receiving the predetermined treatment. In some embodiments, the health states include physiological side effects, post-treatment complications, risks, or other health states.

In some embodiment, the present disclosure comprises means for performing one or more queries based on the predetermined treatment and the social media information associated with the individual to obtain health and social media information associated with similar individuals having (i) similar social media activities and (ii) a record of receiving the predetermined treatment. In some embodiments, such means for performing one or more queries takes the form of communications component 26. In some embodiments, the individual's social media activities are similar to the similar individuals' social media activities prior to receiving the predetermined treatment.

In some embodiments, feature extraction component 28 is configured to extract, from the social media information associated with the population of individuals, one or more personality characteristics, hobbies, personal goals, values, or other attributes associated with each of the population of individuals. In some embodiments, feature extraction component 28 is configured to utilize natural language processing (NLP) to derive the personality characteristics, hobbies, personal goals, values, or other attributes associated with each of the population of individuals from the social media information associated with the population of individuals. In some embodiments, feature extraction component 28 is configured to extract, from the social media information associated with the population of individuals, answers for the BIGS Questionnaire. In some embodiments, the answers provide a scoring over 5 personality traits: (1) Extraversion, (2) Agreeableness, (3) Conscientiousness, (4) Emotional Stability, and (5) Intellect/Imagination. In some embodiments, The BIGS questionnaire may include 50 questions (e.g., 10 questions per personality trait). In some embodiments, feature extraction component 28 is configured such that the questions are scored on a scale from 1 to 5 (i.e., the outcome for each trait is in the range of 5 to 50). In some embodiments, communications component 26 may obtain at least some of the personality characteristics, hobbies, personal goals, values, or other attributes associated with each of the population of individuals from one or more other sources. In some embodiments, where at least some of the foregoing information cannot be extracted from the social media information associated with the population of individuals (or are otherwise not extracted from the social media information), communications component 26 may supplement the extracted information with additional information (e.g., information that could not be extracted from the social media information, but was obtained from one or more other sources).

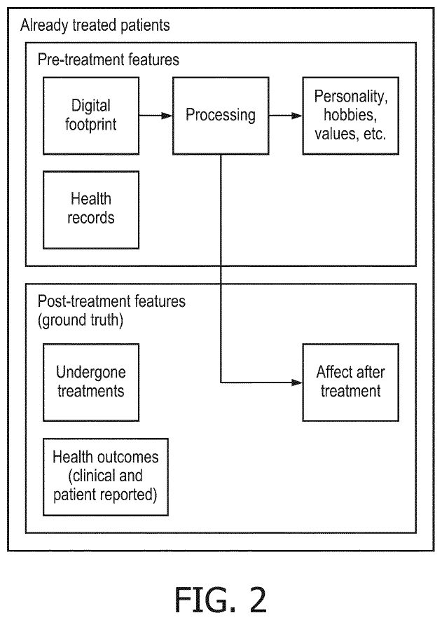

By way of a non-limiting example, FIG. 2 illustrates processing the digital footprint of similar individuals, in accordance with one or more embodiments. As shown in FIG. 2, digital footprint of individuals for whom historical data is available is processed using machine learning and natural language processing techniques. In some embodiments, analysis of the digital footprint (e.g., associated with a population of individuals) yields a clustering of individuals according to their digital footprint (e.g. in terms of hobbies, personality, values, etc.) and comparison of pre-treatment and post-treatment digital footprint yields a list of ways in which the individuals' lives were affected by the predetermined treatment.

Returning to FIG. 1, in some embodiments, feature extraction component 28 is configured to determine, from the social media information associated with the population of individuals, one or more health states associated with the predetermined treatment (e.g., health states related to physical, emotional, or social well-being that are associated with the predetermined treatment). By way of a non-limiting example, Table 1 illustrates a list of health states, in accordance with one or more embodiments. [33]

TABLE 1

| From individuals with similar health | |
|---|---|
| states and similar personal profiles, the | |
| following risk which are most likely to | Excerpts from similar patients digital |
| have high impact on your quality of life: | footprints: |
| Fatigue | ‘so tired after my last radiation session’, |
| | ‘I'm sleeping 12 hours a day since my |
| | treatment’, ‘cancelled my trip to London |
| | because I was not feeling up to it’ |
| Expense of trips to hospitals | ‘can somebody drive me to the hospital, |
| | I can't afford a taxi every time’, ‘on the |
| | bus to the hospital again, wish I could |
| | afford a car’ |
| Living longer | ‘This makes it all worth it, meet my new |
| | grandson!’, ‘Spending some more |
| | quality time with my loving family’, |
| | pictures containing food and people |
| | (social dinners) |

In some embodiments, the personality characteristics, hobbies, personal goals, values, or other attributes associated with each of the population of individuals are used (e.g., by machine learning component 30) to determine a grouping of the social media information associated with the population of individuals to obtain groups representative of a plurality of individuals.

In some embodiments, the personality traits are used to determine the impact on an individual's life. For example, breast cancer treatment may include hormone therapy. As hormone therapy may lead to emotional instability, such a treatment may have a higher impact for an individual who already scores low on emotional stability before treatment. As another example, prostate cancer treatment may include radiation therapy which may lead to rectal toxicity, resulting in bowel problems. As such, an individual, having an extrovert personality, receiving this treatment may need to stay in the vicinity of a bathroom thus less likely to go out. In this example, other extroverted individuals who have received this treatment and experienced this side effect, may feel very unhappy about the health state and may express their dissatisfaction on social media or forums for cancer survivors.

In some embodiments, feature extraction component 28 is configured to determine one or more personal preferences of the individual, the population of individuals, or other individuals. For example, feature extraction component 28 is configured to determine what the individual or other individuals like; whether the individual or other individuals like to go to concerts, watch movies, read books, play sports; or other preferences based on their respective social media information. In a use-case scenario where one of the side effects of the predetermined treatment (e.g. of anesthetics) is diminished capability to concentrate, an individual who enjoys going for a run but does not show interest in reading, is impacted differently from another individual who enjoys reading.

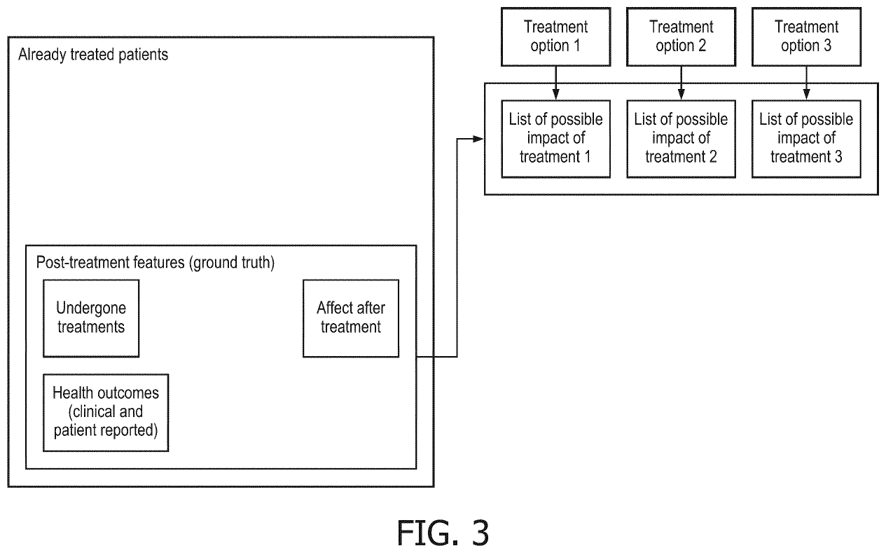

By way of a non-limiting example, FIG. 3 illustrates analysis of post-treatment affect and health outcomes of similar individuals, in accordance with one or more embodiments. As shown in FIG. 3, feature extraction component 28 may identify a list of impacts per treatment based on analysis of health and social media activities of the similar individuals, population of individuals, or other individuals. In some embodiments, feature extraction component 28 is configured to determine treatment impacts by analyzing the features wherein the observed change is the highest for the predetermined treatment and cluster of patients.

Returning to FIG. 1, in some embodiments, each individuals may include one or more characteristics that may not be accurately captured by broad/general population characteristics. As such, a generally trained machine learning model may not accurately predict characteristics of specific individuals. For example, a machine learning model trained on characteristics associated with a population of individuals residing in a country may not accurately predict a specific individual's characteristics who resides in a specific neighborhood in the country.

In some embodiments, the machine learning model may be trained on characteristics associated with individuals (e.g., within the population of individuals) that resemble one or more of the specific individual's characteristics (i.e., similar individuals).

In some embodiments, machine learning component 30 is configured to draw inferences from the data collection by identifying patterns or groupings in the data collection. In some embodiments, one or more clusters are modeled using a measure of similarity. In some embodiments, machine learning component 30 is configured to determine one or more hidden patterns or intrinsic structures in the data collection via unsupervised learning techniques (e.g., clustering algorithms). In some embodiments, the clustering algorithms include one or more of clustering algorithms include: (i) Hierarchical clustering, wherein machine learning component 30 builds a multilevel hierarchy of clusters by creating a cluster tree; (ii) k-Means clustering, wherein machine learning component 30 partitions the data collection into k distinct clusters based on distance to the centroid of a cluster; (iii) Self-organizing maps, wherein machine learning component 30 uses neural networks that learn the topology and distribution of the data; or (iv) other clustering algorithms. For example, machine learning component 30 may be configured to identify segments of the population of individuals with similar attributes. As another example, machine learning component 30 may be configured to identify one or more attributes that separate segments of the population of individuals from each other. As a use-case scenario, machine learning component 30 may provide the data collection (or a portion thereof) as input to a machine learning model (e.g., as described above) to cause the machine learning model to output the group information (e.g., identification of the groups, characteristics of the groups, characteristics of the individuals assigned to the groups, or other information related the groups). In some embodiments, the machine learning model is configured to determine which aspects of the data collection are important. In the context of clustering, the machine learning model determines when to consider two individuals similar or different from each other. In some embodiments, machine learning component 30 is configured to is determine which group an individual identifies with based on the obtained social media information (i.e., related to the individual's social media activities).

In some embodiment, the present disclosure comprises means for providing the health and social media information associated with the similar individuals (e.g., as obtained via communications component 26) to a machine learning model to train the machine learning model. In some embodiments, such means for providing takes the form of machine learning component 30. For example, machine learning component 30 is configured to train the machine learning model on (i) health states, such as survival, side effects, patient reported outcomes, emotional stability (or other mental effects), etc., (ii) post-treatment affective information derived from similar individuals' social media information (i.e., digital footprint), including post-treatment anxiety, ability to execute certain activities, ease with which the inability to execute certain activities was accepted and life goals were reoriented, and (iii) other information.

In some embodiments, machine learning component 30 is configured to generate predictions related to one or more of (a) a likelihood of the health states for the individual, (b) an estimated importance of each of the health states for the individual, or (c) other predictions via the machine learning model (e.g., as described above). As an example, machine learning component 30 may provide, subsequent to the training of the machine learning model, the social media information associated with the individual (or a portion thereof) as input to the machine learning model to cause the machine learning model to output the prediction related to one or more of (a) a likelihood of the health states for the individual, (b) an estimated importance of each of the health states for the individual, or (c) other predictions. In some embodiments, machine learning component 30 is configured to generate predictions related to one or more of (a) a likelihood of the health states for the individual, (b) an estimated importance of each of the health states for the individual, or (c) other predictions based on the individual's identification with the determined group. In some embodiments, machine learning component 30 is configured to generate indications of a need for additional information increase the accuracy of such predictions. As an example, machine learning component 30 may determine one or more features missing from the patient's digital footprint (e.g., that are indicated in the digital footprint of already treated individuals), and provide an indication of such missing features for presentation to a user (e.g., the patient, a caregiver, an administrator, or other user so that the missing features can be obtained from the patient or other sources).

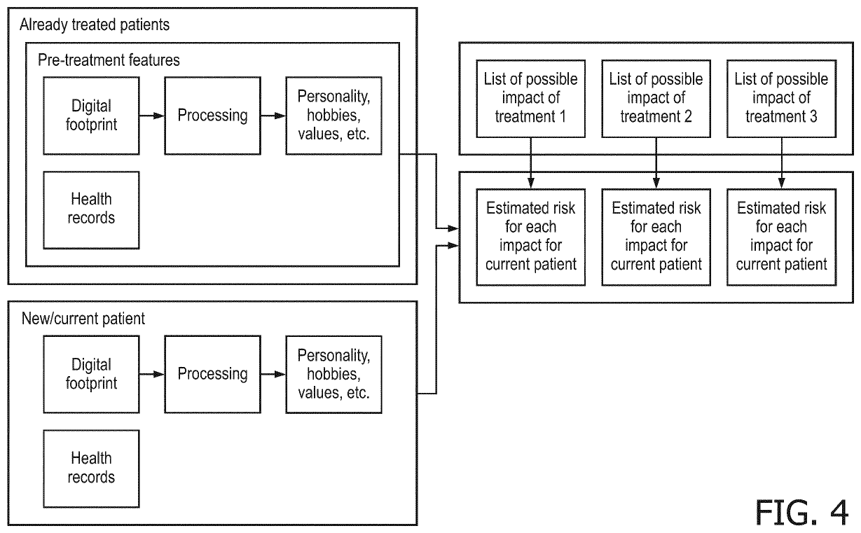

By way of a non-limiting example, FIG. 4 illustrates predicting treatment impacts via a trained machine learning model, in accordance with one or more embodiments. In FIG. 4, a personalized risk of each impact is estimated (e.g., via the machine learning model) for the individual based on similarity to already treated patients (e.g., similar individuals). In this example, the individual's social media activities are provided as input to a machine learning model already trained on health and social media information associated with similar individuals.

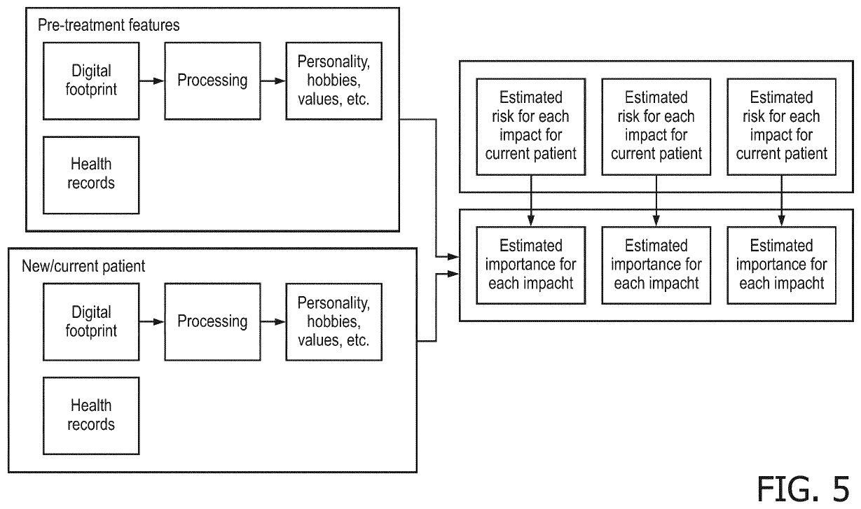

In some embodiments, machine learning component 30 is configured to construct a list of one or more possible future health states after each treatment option based on derived attributes of (past) similar individuals. In some embodiments, machine learning component 30 is configured to determine affective information corresponding to the health states based on a comparison of pre-treatment and post-treatment social media activities of the similar individuals. In some embodiments, machine learning component 30 is configured to estimate the level of impact of each possible future health state on the individual based on a comparison of the social media information associated with the individual and social media information associated with the similar individuals, population of individuals, or other individuals. In some embodiments, machine learning component 30 is configured to identify the health states by analyzing pre-treatment and post-treatment features, wherein the observed change is the highest for the predetermined treatment and cluster of individuals. By way of a non-limiting example, FIG. 5 illustrates estimating the importance of each impact via a trained machine learning model, in accordance with one or more embodiments. As shown in FIG. 5, a machine learning model trained on similar individuals' health records and digital footprint and configured to predict importance of each impact is utilized to predict estimated importance of each impact for the individual.

Returning to FIG. 1, in some embodiments, impact determination component 32 is configured to determine a ranking of the health states that are (i) likely to occur and (ii) likely to have an impact on the individual's life based on a combination of the likelihood of one or more health states and the estimated importance of each of the health states for the individual. In some embodiments, the predicted risk (i.e., likelihood of a health state occurring) is combined with the predicted importance in a function to obtain the ranking. In some embodiments, the function includes determining a product of the predicted risk and the predicted importance. In some embodiments, the function includes adjusting one or more weights associated with the risk and the importance of the health states.

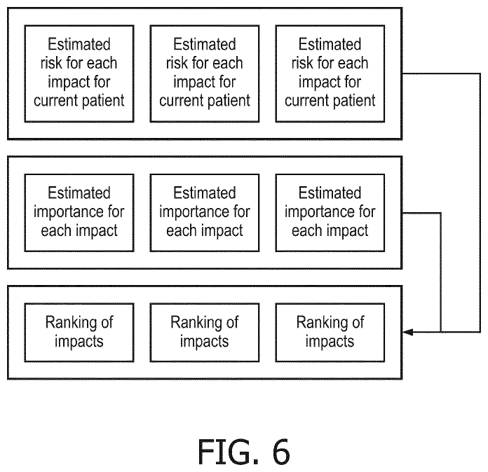

By way of a non-limiting example, FIG. 6 illustrates ranking health states based on a combination of likelihood of occurrence and estimated importance, in accordance with one or more embodiments. As shown in FIG. 6, the likelihood of a health state occurring and the predicted importance of the health state are combined (e.g., multiplied) to determine a ranking of the health states. In some embodiments, the ranking increases proportionally with both likelihood of a health state occurring and importance of a health state.

Returning to FIG. 1, in some embodiments, impact determination component 32 is configured to determine the impact of one or more personal or lifestyle features in quantifications of health-related quality of life. In some embodiments, the lifestyle features include one or more of length of stay in the hospital, number of trips to the hospital; the individual's ability to drive a car to the hospital, whether the individual owns a car, the state of public transportation, the individual's ability to deal with pain, uncertainty, risk, or other health states, important life factors for the individual, the individual's social environment, hobbies, and/or profession, the individual's values and/or beliefs regarding certain aspects (e.g. is the individual religious, need for spirituality), or other personal or lifestyle features. By way of a non-limiting example, Table 2 illustrates risk probability and predicted importance of health states, in accordance with one or more embodiments. [48]

TABLE 2

| Based on patients with similar | | |
|---|---|---|
| health states and similar personal | | Estimated |
| profiles, the following risk | | importance to |
| which are most likely to have high | Risk on a scale | you on a scale |
| impact on your quality of life: | of 0 to 100% | of 0 to 100% |
| Fatigue | 80% | 90% |
| Expense of trips to | 90% | 70% |
| hospitals | | |
| Living longer | 65% | 75% |

For example, for low risk prostate cancer patients, a multitude of different treatment options is available and they all have different risks associated with them. Surgery is more likely to lead to nerve damage resulting in urinary and erectile problems. Radiation is more likely to induce bowel problems and tiredness. Patients are informed of these risks when they are asked to make a treatment decision together with their doctor. However, it may be difficult for people to predict how they will feel about something that might happen in the future. In other words, it may be difficult for patients who have never experienced urinary or bowel problems, how badly they will feel about urinary problems compared to bowel problems in order to make a decision between surgery and radiation. Moreover, if the patients did experience urinary problems before and have bad memories of it, they may overestimate how badly they will feel about urinary problems versus bowel problems, making the decision making process more difficult.

As another example, depression is a common symptom among metastatic breast cancer patients. Depression may develop as a consequence of treatment or disease. In this example, while treatment may prolong life, the quality of life is dependent on how likely the patient is to develop depression and therefore dependent on the patient's personality.

As another example, in advanced stage lung cancer, personal goals such as living long enough to see a grandchild being born, reducing suffering, not wanting to be a (financial) burden for a loved one, or other personal goals may outweigh true physical and mental wellbeing. In some embodiments, these goals or values are not easy to express for patients and a doctor may not be fully aware of the patients personal values in order to assess what is more important for the patient. In such cases, treatment may be postponed such that the patient may, for example, go on an already booked holiday trip.

In yet another example, cardiac patients are often instructed to change their life style; however, changes in lifestyle (e.g. having access to healthy foods, being able to be physically active, etc.) may affect individuals differently. A cook who enjoys food may be happier making larger adjustments in his/her physical activity patterns and in turn allow for smaller adjustments in eating habits, whereas someone who likes to drink wine at parties may be happier to give up fat foods and allow himself/herself to have a glass of wine once a week. In some scenarios, motivation factors to change lifestyles may differ from one person to another. Such motivation factors may include being able to see a grandchild being born, being able to fit in motor suit, feeling more energetic, or other factors may persuade individuals to adhere to their regimens. In this example, an alternative healthy lifestyle prescription or coaching may be the recommended intervention. In some embodiments, lifestyle prescription or coaching may be dependent on personality and personal goals and values associated with the individual.

In some embodiments, the health states are associated with a numerical representation of a reduction in quality of life. In some embodiments, impact determination component 32 is configured to determine the individual's quality adjusted life years based on the numerical representation of the reduction in quality of life and the individual's life expectancy. In some embodiments, quality adjusted life years (QALY) is used to combine quality of life with length of life. In some embodiments, one QALY represents one year of life in “perfect” health. If an individual's health is below this health level, OALYs may be accrued at a rate of less than one per year. In some embodiments, the health states are represented as a utility function. In some embodiments, each health state may include an associated numerical representation of the reduction in quality of life it represents. For example, not being able to use your legs may be represented as a 50% reduction in quality of life, thus having a utility of 0.5. As another example, living with a certain level of pain may be represented as a 25% reduction in quality of life, thus having a utility of 0.75. In yet another example, experiencing anxiety due to a certain illness may be represented as a 10% reduction in quality of life, thus having a utility of 0.9. In some embodiments, an individual's QALY is determined by multiplying each year that he/she is alive by the utility of the health state that he/she is in.

In some embodiments, presentation component 34 is configured to effectuate, via user interface 20, the likelihood of the health states, the estimated importance of each of the health states for the individual, or other information. In some embodiments, presentation component 34 is configured to effectuate, via user interface 20, a predetermined number of top ranked impacts (e.g., top 5 impacts, top 10 impacts) to user 36 (e.g., a health care provider, a doctor, a nurse, the individual) or other users. In some embodiments, presentation component 34 is configured such that ranked impacts are annotated with indications of why they are added (e.g., due to a high risk of occurrence, because it is estimated to be of high importance to the individual). In some embodiments, presentation component 34 is configured to effectuate, via user interface 20, presentation of one or more suggested similar individuals for the individual to connect with (e.g., through social media). For example, if the individual has a question regarding a certain estimated impact, he/she may be directed towards another individual who has experienced the impact. In some embodiments,

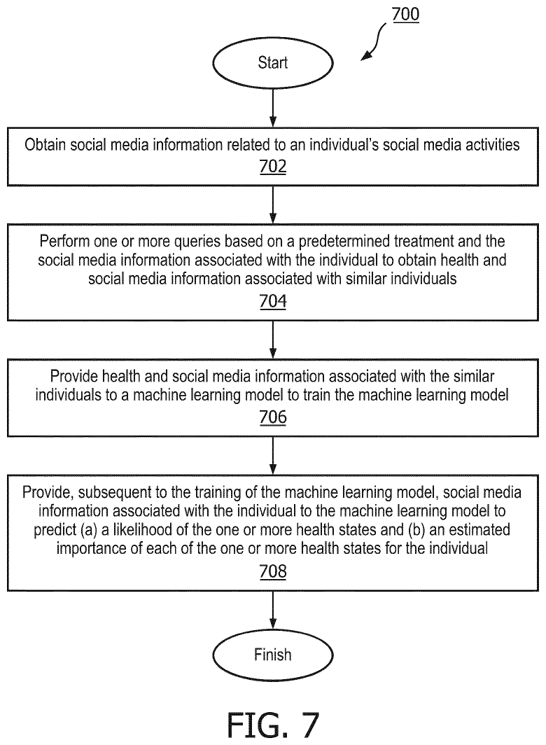

FIG. 7 illustrates a method 700 for providing model-based predictions of quality of life implications of a treatment via individual-specific machine learning models, in accordance with one or more embodiments. Method 700 may be performed with a system. The system comprises one or more processors, or other components. The processors are configured by machine readable instructions to execute computer program components. The computer program components include a communications component, a feature extraction component, a machine learning component, an impact determination component, a presentation component, or other components. The operations of method 700 presented below are intended to be illustrative. In some embodiments, method 700 may be accomplished with one or more additional operations not described, or without one or more of the operations discussed. Additionally, the order in which the operations of method 700 are illustrated in FIG. 7 and described below is not intended to be limiting.

In some embodiments, method 700 may be implemented in one or more processing devices (e.g., a digital processor, an analog processor, a digital circuit designed to process information, an analog circuit designed to process information, a state machine, or other mechanisms for electronically processing information). The devices may include one or more devices executing some or all of the operations of method 700 in response to instructions stored electronically on an electronic storage medium. The processing devices may include one or more devices configured through hardware, firmware, or software to be specifically designed for execution of one or more of the operations of method 700.

At an operation 702, social media information related to an individual's social media activities is obtained from one or more databases. In some embodiments, the individual may be awaiting prescription of a predetermined treatment. In some embodiments, operation 702 is performed by a processor component the same as or similar to communications component 26 (shown in FIG. 1 and described herein).

At an operation 704, one or more queries are performed based on the predetermined treatment and the social media information associated with the individual to obtain health and social media information associated with similar individuals having (i) similar social media activities and (ii) a record of receiving the predetermined treatment. In some embodiments, the health information indicates one or more health states experienced by the similar individuals respectively subsequent to the similar individuals receiving the predetermined treatment. In some embodiments, the social media information associated with the similar individuals indicates one or more personality characteristics, personal goals and values, or other attributes associated with the similar individuals. In some embodiments, operation 704 is performed by a processor component the same as or similar to communications component 26 (shown in FIG. 1 and described herein).

At an operation 706, the health and social media information associated with the similar individuals is provided to a machine learning model to train the machine learning model. In some embodiments, operation 706 is performed by a processor component the same as or similar to machine learning component 30 (shown in FIG. 1 and described herein).

At an operation 708, the social media information associated with the individual is provided to the machine learning model subsequent to the training of the machine learning model to predict (a) a likelihood of the health states and (b) an estimated importance of each of the health states for the individual. In some embodiments, operation 708 is performed by a processor component the same as or similar to machine learning component 30 (shown in FIG. 1 and described herein). [61] Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment. [62] In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A system for providing model-based predictions of quality of life implications of a treatment via individual-specific machine learning models, the system comprising:
one or more processors configured by machine-readable instructions to:
obtain, from one or more databases, social media information related to an individual's social media activities, the individual awaiting prescription of a predetermined treatment;
perform one or more queries based on the predetermined treatment and the social media information associated with the individual to obtain health and social media information associated with similar individuals having (i) similar social media activities and (ii) a record of receiving the predetermined treatment, the health and social media information indicating one or more health states experienced by the similar individuals respectively subsequent to the similar individuals receiving the predetermined treatment;
provide the health and social media information associated with the similar individuals to a machine learning model to train the machine learning model; and
provide, subsequent to the training of the machine learning model, the social media information associated with the individual to the machine learning model to predict (a) a likelihood of the one or more health states and (b) an estimated importance of each of the one or more health states for the individual.

2. The system of claim 1, wherein the individual's social media activities are similar to the similar individuals' social media activities prior to receiving the predetermined treatment.

3. The system of claim 1, wherein the one or more processors are further configured to determine affective information corresponding to the one or more health states based on a comparison of pre-treatment and post-treatment social media activities of the similar individuals.

4. The system of claim 1, wherein the one or more processors are further configured to determine a ranking of the one or more health states that are (i) likely to occur and (ii) likely to have an impact on the individual's life based on a combination of the likelihood of one or more health states and the estimated importance of each of the one or more health states for the individual.

5. The system of claim 4, wherein the one or more health states are associated with a numerical representation of a reduction in quality of life and wherein the one or more processors are further configured to determine the individual's quality adjusted life years based on the numerical representation of the reduction in quality of life and the individual's life expectancy.

6. The system of claim 1, wherein the individual's social media activities and/or the similar individuals' social media activities comprise one or more of Facebook likes, Facebook posts, Tweets, Retweets, accounts followed on Twitter, people followed on LinkedIn, browsing data, or forum posts.

7. A method for providing model-based predictions of quality of life implications of a treatment via individual-specific machine learning models, the method comprising:
obtaining, with one or more processors, social media information related to an individual's social media activities from one or more databases, the individual awaiting prescription of a predetermined treatment;
performing, with the one or more processors, one or more queries based on the predetermined treatment and the social media information associated with the individual to obtain health and social media information associated with similar individuals having (i) similar social media activities and (ii) a record of receiving the predetermined treatment, the health and social media information indicating one or more health states experienced by the similar individuals respectively subsequent to the similar individuals receiving the predetermined treatment;
providing, with the one or more processors, the health and social media information associated with the similar individuals to a machine learning model to train the machine learning model; and
providing, with the one or more processors, the social media information associated with the individual to the machine learning model subsequent to the training of the machine learning model to predict (a) a likelihood of the one or more health states and (b) an estimated importance of each of the one or more health states for the individual.

8. The method of claim 7, wherein the individual's social media activities are similar to the similar individuals' social media activities prior to receiving the predetermined treatment.

9. The method of claim 7, further comprising determining, with the one or more processors, affective information corresponding to the one or more health states based on a comparison of pre-treatment and post-treatment social media activities of the similar individuals.

10. The method of claim 7, further comprising determining, with the one or more processors, a ranking of the one or more health states that are (i) likely to occur and (ii) likely to have an impact on the individual's life based on a combination of the likelihood of one or more health states and the estimated importance of each of the one or more health states for the individual.

11. The method of claim 10, wherein the one or more health states are associated with a numerical representation of a reduction in quality of life and wherein the method further comprises determining, with the one or more processors, the individual's quality adjusted life years based on the numerical representation of the reduction in quality of life and the individual's life expectancy.

12. The method of claim 7, wherein the individual's social media activities and/or the similar individuals' social media activities comprise one or more of Facebook likes, Facebook posts, Tweets, Retweets, accounts followed on Twitter, people followed on LinkedIn, browsing data, or forum posts.

13. A system for providing model-based predictions of quality of life implications of a treatment via individual-specific machine learning models, the system comprising:
means for obtaining social media information related to an individual's social media activities from one or more databases, the individual awaiting prescription of a predetermined treatment;
means for performing one or more queries based on the predetermined treatment and the social media information associated with the individual to obtain health and social media information associated with similar individuals having (i) similar social media activities and (ii) a record of receiving the predetermined treatment, the health and social media information indicating one or more health states experienced by the similar individuals respectively subsequent to the similar individuals receiving the predetermined treatment;
means for providing the health and social media information associated with the similar individuals to a machine learning model to train the machine learning model; and
means for providing the social media information associated with the individual to the machine learning model subsequent to the training of the machine learning model to predict (a) a likelihood of the one or more health states and (b) an estimated importance of each of the one or more health states for the individual.

14. The system of claim 13, wherein the individual's social media activities are similar to the similar individuals' social media activities prior to receiving the predetermined treatment.

15. The system of claim 13, further comprising means for determining affective information corresponding to the one or more health states based on a comparison of pre-treatment and post-treatment social media activities of the similar individuals.

16. The system of claim 13, further comprising means for determining a ranking of the one or more health states that are (i) likely to occur and (ii) likely to have an impact on the individual's life based on a combination of the likelihood of one or more health states and the estimated importance of each of the one or more health states for the individual.

17. The system of claim 16, wherein the one or more health states are associated with a numerical representation of a reduction in quality of life and wherein the system further comprises means for determining the individual's quality adjusted life years based on the numerical representation of the reduction in quality of life and the individual's life expectancy.

18. The system of claim 13, wherein the individual's social media activities and/or the similar individuals' social media activities comprise one or more of Facebook likes, Facebook posts, Tweets, Retweets, accounts followed on Twitter, people followed on LinkedIn, browsing data, or forum posts.
